(19) United States
(12) Patent Application Publication
(10) Pub. No.: US 2021/0293831 A1
(43) Pub. Date: Sep. 23, 2021

(54) Method of Performing Differential Diagnosis of Neurodegenerative Diseases in a Subject

(71) Applicant: ALZOHIS, Paris (FR)

(72) Inventors: Romain Verpillot, Paris (FR); Hervé Thirlez, Versailles (FR)

(73) Assignee: ALZOHIS, Paris (FR)

(21) Appl. No.: 17/261,707

(22) PCT Filed: Jul. 22, 2019
(86) PCT No.: PCT/EP2019/069675

§ 371 Date: Jan. 20, 2021

(30) Foreign Application Priority Data
Jul. 20, 2018 (LU) LU100877
Apr. 25, 2019 (LU) LU101191

(51) Int. Cl. G01N 33/68; G01N 33/94; G16H 10/20; G16H 20/70; G16H 50/20; G16H 50/30

(52) U.S. Cl. CPC G01N 33/6896; G01N 33/9413; G01N 33/9433; G01N 2800/2821; G16H 20/70; G16H 50/20; G16H 50/30; G16H 10/20

(57) ABSTRACT

The invention is directed to an in vitro method for performing a differential diagnosis of neurodegenerative diseases in a subject, said subject being selected among subjects suffering from Alzheimer's disease, mental depression, dementia with Lewy Body, frontotemporal dementia, and/or vascular dementia. Said method comprises the steps of: (a) determining at least five criteria of said subject, (b) comparing said at least five criteria of said subject with reference values by calculating a global note in relation with each neurodegenerative disease, and (c) determining whether said subject suffers from Alzheimer's disease, mental depression, dementia with Lewy Body, frontotemporal dementia, vascular dementia or mixed dementia.

### TECHNICAL FIELD

The invention is directed to a method for performing a differential diagnosis of neurodegenerative diseases in a subject.

### BACKGROUND ART

The performance of a differential diagnosis allows a clinician to categorize the patient, who has then the opportunity to follow an adequate treatment in order to heal, or at least to improve his/her medical conditions.

The neurodegenerative diseases, which are characterized by the progressive loss of structure or function of neurons, are numerous, as shown by the following list: Alzheimer's disease, mental depression, amyotrophic lateral sclerosis, frontotemporal dementia, Parkinson's disease, progressive supranuclear palsy, Parkinson's disease with dementia, dementia with Lewy Body, vascular dementia, etc.

With regard with Alzheimer's disease, it has been noticed that an elevated concentration of noradrenaline can be an etiological for some patients (Fitzgerald P. J., Current Alzheimer Research, 2010, 7, 506-516).

The frontiers between those diseases are sometimes difficult to distinguish, especially in the case of Alzheimer's disease so that the clinician in charge of the follow-up of the patients can hesitate and give an inappropriate treatment to one patient. The diagnosis can also be difficult to establish in the sense that it implies expensive and cumbersome experiments, such as medical imaging and/or lumbar punctures to collect a sample of cerebrospinal (CSF) fluid. It is also noted that the absence of syndrome does not “rule out” the presence of a disease. There are up to 30% misdiagnosis without biomarkers (Jack C. R., et al., Alzheimer's & Dementia, 2018, 14, 532-562).

On the other hand, medical questionnaires are not sufficient for the clinician to be certain of the diagnosed disease.

A link between morphological and functional changes occurring in the monoaminergic ascending system and the physiopathology of AD has been suggested (Simic G. et al., Neuropathol. Appl. Neurobiol., 2009, 35, 532-554 and Trillo L. et al., Neurosci Biobehav. Rev., 2013, 37, 1363-1379). Noradrenergic neurons in the locus coeruleus (LC) are the main source of noradrenergic inputs to numerous regions throughout the brain. Due to its widespread efferent innervation, the LC projection system plays a pivotal regulatory role in processes such as stress and maintenance of cognitive performances (Sara S. J., Nat. Rev. Neurosci., 2009, 10, 211-223). This leads to the quest of catecholamines detection in order to develop new tools for neurodegenerative disease diagnosis. Catecholamines are monoamines derived from tyrosine and play the role of neuromodulators in the central nervous system and in the peripheral sympathetic nervous system, as well as hormones in the blood circulation.

Mixed dementia is also not very well treated since the patients are often diagnosed with just a single type of dementia. In this case, a physician will base the pharmaceutical decisions/prescriptions on the type of dementia that has been diagnosed, which at the end, will not help the patient to improve his/her living conditions.

### SUMMARY OF INVENTION

### Technical Problem

The invention has for technical problem to alleviate at least one of the drawbacks present in the prior art. More particularly, the invention has for technical problem to avoid expensive and cumbersome experiments to diagnose a subject suffering from a neurodegenerative disease or from a mixed dementia. Said disease can be one of the following: Alzheimer's disease, mental depression, dementia with Lewy Body, frontotemporal dementia, vascular dementia or mixed dementia.

### Technical Solution

The invention is directed to an in vitro method for performing a differential diagnosis of neurodegenerative diseases in a subject, said subject being selected among subjects suffering from Alzheimer's disease, mental depression, dementia with Lewy Body, frontotemporal dementia, and/or vascular dementia. Said method comprises the steps of: (a) determining at least five criteria of said subject, (b) comparing said at least five criteria of said subject with reference values by calculating a global note in relation with each neurodegenerative disease, and (c) determining whether said subject suffers from Alzheimer's disease, mental depression, dementia with Lewy Body, frontotemporal dementia, vascular dementia or mixed dementia.

According to a preferred embodiment, said subject suffering from Alzheimer's disease is selected among subjects suffering from Alzheimer's disease with a noradrenaline concentration [NA] comprised between 3000 pmol/l and 15000 pmol/l and among subjects suffering from Alzheimer's disease with a noradrenaline concentration [NA] comprised between 100 pmol/l and 2700 pmol/l.

According to a preferred embodiment, said step (a) is a step of determining at least eight criteria.

According to a preferred embodiment, said criteria are selected from
an age of said subject,
a score of said subject to a questionnaire adapted for screening cognitive function, preferentially a mini-mental state examination questionnaire,
a dopamine concentration [D] in one blood sample of said subject,
an adrenaline concentration [A] in one blood sample of said subject,
a noradrenaline concentration [NA] in one blood sample of said subject,
a dopamine expected concentration [D]^{expected} in one blood sample of said subject,
an adrenaline expected concentration [A]^{expected} in one blood sample of said subject,
a noradrenaline expected concentration [NA]^{expected} in one blood sample of said subject.

According to a preferred embodiment said blood sample of said subject is one plasma sample of said subject.

According to a preferred embodiment, said dopamine, adrenaline and noradrenaline concentrations are determined with an HPLC equipped with an electrochemical detector.

According to a preferred embodiment, said dopamine expected concentration is obtained by applying the following equation

[D]^{expected}=min(K₁;|[D]−[̅D̅]̅_̅{̅A̅l̅z̅h̅e̅i̅m̅e̅r̅}̅|)
wherein K₁ is a constant comprised between 5 and 25, and
wherein [D]_{Alzheimer} is 454 pmol/l.

According to a preferred embodiment, said adrenaline expected concentration is obtained by applying the following equation

[A]^{expected}=min(K₁;|[A]−[̅A̅]̅_̅{̅A̅l̅z̅h̅e̅i̅m̅e̅r̅}̅|)
wherein K₁ is a constant comprised between 5 and 25, and
wherein [A]_{Alzheimer} is 483 pmol/l.

According to a preferred embodiment, said noradrenaline expected concentration is obtained by applying the following equation

[NA]^{expected}=min(K₁;|[NA]−[̅N̅A̅]̅_̅{̅A̅l̅z̅h̅e̅i̅m̅e̅r̅}̅|)
wherein K₁ is a constant comprised between 5 and 25, and
wherein [NA]_{Alzheimer} is 4339 pmol/l.

According to a preferred embodiment, said reference values used in step (b) are as follows:
a. the age of said subject is comprised within the range 40.2 years and 89.8 years,
b. the score of said questionnaire adapted for screening cognitive function is comprised between 0 and 30,
c. the dopamine concentration is comprised between 111 pmol/l and 1334 pmol/l,
d. the adrenaline concentration is comprised between 66 pmol/l and 2366 pmol/l,
e. the noradrenaline concentration is comprised between 874 pmol/l and 8681 pmol/l,
f. the dopamine expected concentration is comprised between 2 pmol/l and 838 pmol/l,
g. the adrenaline expected concentration is comprised between 8 pmol/l and 1788 pmol/l,
h. the noradrenaline expected concentration is comprised between 2 pmol/l and 4727 pmol/l.

According to a preferred embodiment, said method further comprises the step of attributing a correction factor δ to said reference values, said correction factor δ being comprised between 0.50 and 1.50.

According to a preferred embodiment, said step (b) of calculating a global note in relation with each neurodegenerative disease comprises the following sub-steps:
a) determining a virtual value V_{disease}^{criterion} by neurodegenerative disease in function of each criterion,
b) attributing a weighting factor to each virtual value V_{disease}^{criterion} determined in the previous sub-step,
c) determining a sum of each of said weighted virtual values so as to obtain one global note in relation with each neurodegenerative disease.

According to a preferred embodiment, said virtual value V_{disease}^{criterion} by neurodegenerative disease in function of each criterion is zero if one measured value VAL^{criterion} of said subject for each criterion is outside the range of the reference values defined above, or said virtual value V_{disease}^{criterion} by neurodegenerative disease in function of each criterion is determined for each criterion by applying the following equation if the measured value VAL^{criterion} of said subject for each criterion is within the range of the reference values defined above:

$${V}_{d i s e a s e}^{c r i t e r i o n} = 1 + K \frac{V A {L}^{criterion} - {MINI}_{dise ase}^{criterion}}{{MAXI}_{disease}^{criterion} - {MINI}_{dise ase}^{criterion}}$$

wherein VAL^{criterion} is the measured value of one subject, said measured value corresponding to each criterion, said criterion being defined above,
wherein MINI_{disease}^{criterion} is the minimum reference value with respect to each neurodegenerative disease in function of each criterion,
wherein MAXI_{disease}^{criterion} is the maximum reference value with respect to each neurodegenerative disease in function of each criterion, and
wherein K is a mathematical parameter, K being preferentially comprised between 0 and 0.1.

According to a preferred embodiment, the weighting factor (w₁) in relation with the virtual value with respect to the age of said subject and to the score of said subject is comprised between 0.1 and 0.9, preferentially is equal to 0.5 and the weighting factor (w₂) in relation with the virtual value with respect to the concentrations and the expected concentrations comprised between 1.1 and 3.0, preferentially is equal to 2.

According to a preferred embodiment, said step of determining whether said subject suffers from Alzheimer's disease, mental depression, dementia with Lewy Body, frontotemporal dementia, vascular dementia or mixed dementia is performed by comparing the global notes obtained in relation with each of neurodegenerative diseases.

According to a preferred embodiment, said method further comprises, between said step (c) and said step (d), a step (c′) of determination whether said subject suffers from arterial hypertension.

In general, the particular embodiments of each object of the invention are also applicable to other objects of the invention. To the extent possible, each object of the invention is combinable with other objects.

According to a further aspect, the invention has for object an in vitro method for performing a differential diagnosis of neurodegenerative diseases selected from early stage of Alzheimer's disease (AD1), advanced stage of Alzheimer's disease (AD2), mental depression (PSY), dementia with Lewy bodies (DLB), frontotemporal dementia (FTD), and/or vascular dementia (VD); said method being performed in a subject presenting signs of dementia;

said method comprising the steps of:

a) determining at least five criteria of said subject, said at least five criteria comprising the age of said subject, a score of said subject to a questionnaire adapted for screening cognitive function, a dopamine concentration [D] in a blood sample of said subject, an adrenaline concentration [A] in said blood sample of said subject and a noradrenaline concentration [NA] in said blood sample of said subject,

c) calculating one global note per each neurodegenerative disease according to the following sub-steps:

i) determining for one neurodegenerative disease one parameter V_{disease}^{criterion} per criterion, said parameter combining one criterion selected from the five criteria determined in step (a) with reference values of said one criterion in function of said one neurodegenerative disease,

ii) summing each parameter V_{disease}^{criterion} related to said one neurodegenerative disease obtained in sub-step (i) so as to obtain one global note in relation with said one neurodegenerative disease;

iii) repeating sub-steps (i) and (ii) for each neurodegenerative disease,

d) determining, based on each global note per neurodegenerative disease calculated in steps (c), whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD);

said method being remarkable in that it further comprises:
a step (b) performed after step (a); wherein step (b) comprises the step of comparing said noradrenaline concentration [NA] to a predetermined threshold, and if said noradrenaline concentration [NA] is lower or equal to said predetermined threshold, then determining the arterial hypertension status of said subject,
and in that step (c) further comprises, if said noradrenaline concentration [NA] is lower or equal to said predetermined threshold, the step of calculating the global note in relation with advanced stage of Alzheimer's disease (AD2) by adding a parameter V^{HT} in relation with the arterial hypertension status of said subject.

Surprisingly, the inventors have found that it is possible to provide a precise diagnosis of neurodegenerative diseases by the analysis of at least five criteria of a subject, wherein the five criteria are combined together with reference values characteristic of each of the following neurodegenerative diseases selected from early stage of Alzheimer's disease (AD1), advanced stage of Alzheimer's disease (AD2), mental depression (PSY), dementia with Lewy bodies (DLB), frontotemporal dementia (FTD) and/or vascular dementia (VD). The unique combination of the five criteria and the additional criterion related to the arterial hypertension status followed by the determination of a global note allows for providing a method of diagnosis that can determine whether the subject suffers from AD, mental depression, DLB, FTD, VD and/or mixed dementia.

It is further notable that the diagnosis is not invasive and is performed in vitro on a blood sample easily collected (by comparison with a lumbar puncture for example) from the subjects. Moreover, this allows for the repeatability of the diagnosis and thus for statistical confirmation of the diagnosis.

The accuracy of the differential diagnosis is such that it can also diagnose between two distinct stages of Alzheimer's disease, namely an early stage of Alzheimer's disease and an advanced stage of Alzheimer's disease.

Such differential diagnosis allows the categorization of the subject according to her/his disease and this is an opportunity for such a person to receive adequate medical treatments. Moreover, the diagnosis allows for identifying subjects who are suffering from mixed dementia, which is not easy since such type of subjects can show symptoms that are relevant for two or more neurodegenerative diseases.

The following features of the in vitro method for performing a differential diagnosis of neurodegenerative disease in one subject are advantageously used to better define the present invention.
Said subject is in homeostatic imbalance.
The predetermined threshold of step (b) is a noradrenaline concentration [NA] comprised between 2900 pmol/l and 3100 pmol/l, preferentially comprised between 2950 pmol/l and 3050 pmol/l, more preferentially comprised between 2975 pmol/l and 3025 pmol/l, even more preferentially is a noradrenaline concentration [NA] of at least 3000 pmol/l.
In one instance, the predetermined threshold is a noradrenaline concentration [NA] of 3023 pmol/l.
Said steps (b), (c) and (d) are carried out by a computer.
The questionnaire adapted for screening cognitive function is a mini-mental state examination (MMSE) questionnaire.
Said parameter V_{disease}^{criterion} per criterion in function of each neurodegenerative disease is determined in sub-step (i) according to the following mathematical equation (1):

$${V}_{d i s e a s e}^{c r i t e r i o n} = 1 + K \frac{V A {L}^{criterion} - {MINI}_{dise ase}^{criterion}}{{MAXI}_{disease}^{criterion} - {MINI}_{dise ase}^{criterion}} \qquad ( 1 )$$

wherein VAL^{criterion} is the measured value of one of the five criteria determined in step (a), wherein MINI_{disease}^{criterion} is the minimum reference value with respect to each neurodegenerative disease in function of each criterion, wherein MAXI_{disease}^{criterion} is the maximum reference value with respect to each neurodegenerative disease in function of each criterion, and wherein K is a mathematical parameter, K being comprised between 0.01 and 1.00, preferentially between 0.02 and 0.99, more preferentially between 0.05 and 0.95, even more preferentially between 0.10 and 0.90.
The provision that if VAL^{criterion}<MINI_{disease}^{criterion} or if VAL^{criterion} MAXI_{disease}^{criterion} then V=0.
Said in vitro method for performing a differential diagnosis of neurodegenerative disease in one subject further comprises the step of attributing a weighting factor to each parameter V_{disease}^{criterion}.
Said weighting factor is comprised between 1.0 and 10.0 with respect to one or more parameters selected from the parameter in relation with the age of said subject, the parameter in relation with the dopamine concentration [D], the parameter in relation with the adrenaline concentration [A] and the parameter in relation with the noradrenaline concentration [NA]; preferentially the weighting factor is 5.0.
Said weighting factor is comprised between 0.1 and 3.0 with respect to the parameter in relation with the score to a questionnaire adapted for screening cognitive function of said subject; preferentially the weighting factor with respect to the parameter in relation with the score to a questionnaire adapted for screening cognitive function of said subject is 2.0.
Said parameter V^{HT} in relation with the arterial hypertension status of said subject is function of the age of said subject, and is comprised between 0 and 11.0; preferentially is 3.0.
Said parameter V^{HT} in relation with the arterial hypertension status of said subject is determined according to the following mathematical equation (2) V^{HT}=α(age)+β (2) wherein α is a variable in function of the age of said subject; with preference α is a value comprised between 0.0 and 10.0, wherein β is a variable in function of the seriousness of the arterial hypertension status; with preference β is a value comprised between 0.0 and 1.0.
Said in vitro method for performing a differential diagnosis of neurodegenerative disease in one subject comprises the step of applying a correction factor □ to said reference values, said correction factor Q being comprised between 0.50 and 1.50, preferentially between 0.60 and 1.40.
Said blood sample of said subject is one plasma sample of said subject.
Said noradrenaline, dopamine and adrenaline concentrations are determined with an HPLC equipped with an electrochemical detector.
Said step (d) of determining whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD) is performed by comparing the global notes obtained in relation with each of neurodegenerative diseases.
Said step (d) of determining whether said subject suffers from one or more neurodegenerative diseases selected from early stage Alzheimer's disease (AD1), advanced stage of Alzheimer's disease (AD2), mental depression (PSY), dementia with Lewy bodies (DLB), frontotemporal dementia (FTD), and/or vascular dementia (VD) is equivalent to a step of determining whether said subject suffers from one neurodegenerative disease selected from early stage Alzheimer's disease (AD1), advanced stage of Alzheimer's disease (AD2), mental depression (PSY), dementia with Lewy bodies (DLB), frontotemporal dementia (FTD), or vascular dementia (VD) or whether said subject suffers from mixed dementia.
Said step (a) further comprises the determination of one or more of the following criteria, said criteria being the sex of said subject or a presence of an APOE□4 allele in the genotype of said subject.

In a preferred embodiment, the reference values used in step (c) in relation with early stage of Alzheimer's disease (AD1) are as followed:
with respect to the age of said subject, the minimum reference value is at least 50 years, preferably is 52.92; and/or the maximum reference value is at most 90 years; preferably is 87.72 years;
with respect to the score of said subject to a questionnaire adapted for screening cognitive function, the minimum reference value is at least 14, preferably is 15; and/or the maximum reference value is at most 29, preferably is 28;
with respect to the dopamine concentration [D], the minimum reference value is at least 90 pmol/l, preferably is 110.74 pmol/l; and/or the maximum reference value is at most 1000 pmol/l, preferably is 878.22 pmol/l;
with respect to the adrenaline concentration [A] the minimum reference value is at least 40 pmol/l, preferably is 65.66 pmol/l; and/or the maximum reference value is at most 2000 pmol/l, preferably is 1720.74 pmol/l;
with respect to the noradrenaline concentration [NA] the minimum reference value is at least 2900 pmol/l, preferably is 2962.54 pmol/l; and/or the maximum reference value is at most 9000 pmol/l, preferably is 8681.22 pmol/l.

In a preferred embodiment, the reference values used in step (c) in relation with advanced stage of Alzheimer's disease (AD2) are as followed:
with respect to the age of said subject, the minimum reference value is at least 45 years, preferably is 51.94; and/or the maximum reference value is at most 95 years, preferably is 89.76 years;
with respect to the score of said subject to a questionnaire adapted for screening cognitive function, the minimum reference value is at least 3, preferably is 4; and/or the maximum reference value is at most 30, preferably is 29;
with respect to the dopamine concentration [D], the minimum reference value is at least 140 pmol/l, preferably is 152.88 pmol/l; and/or the maximum reference value is at most 1000 pmol/l, preferably is 655.86 pmol/l;
with respect to the adrenaline concentration [A] the minimum reference value is at least 80 pmol/l, preferably is 143.08 pmol/l; and/or the maximum reference value is at most 1500 pmol/l, preferably is 1371.09 pmol/l;
with respect to the noradrenaline concentration [NA] the minimum reference value is at least 750 pmol/l, preferably is 1023.12 pmol/l; and/or the maximum reference value is at most 3000 pmol/l, preferably is 2758.08 pmol/l.

In a preferred embodiment, the reference values used in step (c) in relation with mental depression (PSY) are as followed:
with respect to the age of said subject, the minimum reference value is at least 35 years, preferably is 40.18; and/or the maximum reference value is at most 80 years, preferably is 74.46 years;
with respect to the score of said subject to a questionnaire adapted for screening cognitive function, the minimum reference value is at least 21, preferably is 22; and/or the maximum reference value is at most 30, preferably is 29;
with respect to the dopamine concentration [D], the minimum reference value is at least 250 pmol/l, preferably is 276.36 pmol/l; and/or the maximum reference value is at most 750 pmol/l, preferably is 519.18 pmol/l;
with respect to the adrenaline concentration [A] the minimum reference value is at least 180 pmol/l, preferably is 191.10 pmol/l; and/or the maximum reference value is at most 450 pmol/l, preferably is 406.98 pmol/l;
with respect to the noradrenaline concentration [NA] the minimum reference value is at least 1000 pmol/l, preferably is 1533.70 pmol/l; and/or the maximum reference value is at most 4000 pmol/l, preferably is 3691.38 pmol/l.

In a preferred embodiment, the reference values used in step (c) in relation with dementia with Lewy bodies (DLB) are as followed:
with respect to the age of said subject, the minimum reference value is at least 55 years, preferably is 61.74; and/or the maximum reference value is at most 85 years, preferably is 81.6 years;
with respect to the score of said subject to a questionnaire adapted for screening cognitive function, the minimum reference value is at least 7, preferably is 8; and/or the maximum reference value is at most 25, preferably is 24;
with respect to the dopamine concentration [D], the minimum reference value is at least 150 pmol/l, preferably is 203.84 pmol/l; and/or the maximum reference value is at most 400 pmol/l, preferably is 375.36 pmol/l;
with respect to the adrenaline concentration [A] the minimum reference value is at least 80 pmol/l, preferably is 103.88 pmol/l; and/or the maximum reference value is at most 700 pmol/l, preferably is 418.20 pmol/l;
with respect to the noradrenaline concentration [NA] the minimum reference value is at least 900 pmol/l, preferably is 1065.26 pmol/l; and/or the maximum reference value is at most 8000 pmol/l, preferably is 7216.50 pmol/l.

In a preferred embodiment, the reference values used in step (c) in relation with frontotemporal dementia (FTD) are as followed:
with respect to the age of said subject, the minimum reference value is at least 45 years, preferably is 49; and/or the maximum reference value is at most 80 years, preferably is 75.48 years;
with respect to the score of said subject to a questionnaire adapted for screening cognitive function, the minimum reference value is at least 0, preferably is 0; and/or the maximum reference value is at most 28, preferably is 27;
with respect to the dopamine concentration [D], the minimum reference value is at least 200 pmol/l, preferably is 245.98 pmol/l; and/or the maximum reference value is at most 1100 pmol/l, preferably is 1002.66 pmol/l;
with respect to the adrenaline concentration [A] the minimum reference value is at least 100 pmol/l, preferably is 123.48 pmol/l; and/or the maximum reference value is at most 1500 pmol/l, preferably is 1270.92 pmol/l;
with respect to the noradrenaline concentration [NA] the minimum reference value is at least 700 pmol/l, preferably is 874.16 pmol/l; and/or the maximum reference value is at most 6000 pmol/l; preferably is 5743.62 pmol/l.

In a preferred embodiment, the reference values used in step (c) in relation with vascular dementia (VD) are as followed:
with respect to the age of said subject, the minimum reference value is at least 55 years, preferably is 58.8; and/or the maximum reference value is at most 90 years, preferably is 85.68 years;
with respect to the score of said subject to a questionnaire adapted for screening cognitive function, the minimum reference value is at least 20, preferably is 21; and/or the maximum reference value is at most 29, preferably is 28;
with respect to the dopamine concentration [D], the minimum reference value is at least 140 pmol/l, preferably is 198.94 pmol/l; and/or the maximum reference value is at most 580 pmol/l, preferably is 533.46 pmol/l;
with respect to the adrenaline concentration [A] the minimum reference value is at least 200 pmol/l, preferably is 240.10 pmol/l; and/or the maximum reference value is at most 1800 pmol/l, preferably is 1525.92 pmol/l;
with respect to the noradrenaline concentration [NA] the minimum reference value is at least 1400 pmol/l, preferably is 1518.02 pmol/l; and/or the maximum reference value is at most 4000 pmol/l; preferably is 3705.66 pmol/l.

In a preferred embodiment, the step (c) further comprises calculating one global note in relation with neurological control (NC); with preference, the reference values used in step (c) in relation with neurological control (NC) are as followed:
with respect to the age of said subject, the minimum reference value is at least 35 years, preferably is 40.18; and/or the maximum reference value is at most 90 years, preferably is 84.66 years;
with respect to the score of said subject to a questionnaire adapted for screening cognitive function, the minimum reference value is at least 22, preferably is 23; and/or the maximum reference value is at most 30, preferably is 30;
with respect to the dopamine concentration [D], the minimum reference value is at least 180 pmol/l, preferably is 207.76 pmol/l; and/or the maximum reference value is at most 1600 pmol/l, preferably is 1334.16 pmol/l;
with respect to the adrenaline concentration [A] the minimum reference value is at least 110 pmol/l, preferably is 163.66 pmol/l; and/or the maximum reference value is at most 2500 pmol/l, preferably is 2366.40 pmol/l;
with respect to the noradrenaline concentration [NA] the minimum reference value is at least 900 pmol/l, preferably is 925.12 pmol/l; and/or the maximum reference value is at most 7000 pmol/l; preferably is 6331.14 pmol/l.

According to a further aspect, the invention has for object a data processing system comprising a processor configured to perform the method in accordance with the first aspect when said steps (b), (c) and (d) are carried out by a computer.

According to a further aspect, the invention has for object a computer program comprising instructions which, when the program is executed by a computer, cause the computer to carry out the method in accordance with the first aspect when said steps (b), (c) and (d) are carried out by the computer.

According to a further aspect, the invention has for object a computer-readable data carrier having stored thereon the computer program in accordance with the third aspect of the invention.

### Advantages of the Invention

The invention is particularly interesting in that the differential diagnosis of a patient, when said diagnosis is precise, allows the patient to be adequately followed-up by the clinician. Once the patient has been diagnosed with the correct disease she/he suffers from, she/he can receive the best medical care that is going to help her/him to improve her/his medical conditions. While the differential diagnosis allows for detection of a mixed dementia, it is also an advantage to provide adequate “mixed treatment” for responding more efficacy to such health conditions, especially since it is currently difficult to detect such mixed dementia conditions and to know which disease is implicated when a mixed dementia condition is detected.

The use of blood test allows for the ease of repeatability of the diagnosis, allowing the patient to be followed up, to be diagnosed early (and it is known than an early diagnosis favours the treatment and greatly improves the condition of life of the patient and his/her family).

### BRIEF DESCRIPTION OF THE DRAWINGS

FIG. 1: Principles of the method of the present invention.

FIG. 2: Distribution of the subjects in the panel used in the determination of the method.

FIG. 3: Cloud of points using the global note from the point of view from the group suffering from Alzheimer's disease with a noradrenaline concentration [NA] comprised between 3000 pmol/l and 15000 pmol/l (AD1).

FIG. 4: Cloud of points using the global note from the point of view from the group suffering from Alzheimer's disease with a noradrenaline concentration [NA] comprised between 100 pmol/l and 2700 pmol/l (AD2).

FIG. 5 shows the histogram distribution of the four groups of subjects: AD1, AD2, FTD/DLB/VD and PSY/NC.

FIG. 6 shows the relative frequency distribution of plasma [NA] concentrations of AD (n=50) and non-AD (n=50) subjects.

FIG. 7 shows the Receiver Operating Characteristic (ROC) curve of plasma [NA] concentrations in AD versus non-AD subjects.

### DESCRIPTION OF AN EMBODIMENT

The present invention has allowed for the development of an efficient differential diagnosis method.

The diseases that are diagnosed thanks to the method of the present invention are the following neurodegenerative diseases: from Alzheimer's disease with a noradrenaline concentration [NA] comprised between 3000 pmol/l and 15000 pmol/l (AD1), Alzheimer's disease with a noradrenaline concentration [NA] comprised between 100 pmol/l and 2700 pmol/l (AD2), mental depression (PSY), dementia with Lewy Body (DLB), frontotemporal dementia (FTD), and/or vascular dementia (VD). The diagnosis is able to discriminate which neurodegenerative disease a patient is suffering from. The diagnosis method of the present invention also evidently allows for discriminating patients suffering from mild cognitive impairment. Such patients typically present cognitive problems, anxiety, burn-out, sleep apnea, alcoholism. They are refers as control patient or neurocontrol patient (Control/CTRL/NeuroControl/NeuroCtrl).

The sensitivity, which is the percentage of patients suffering from the disease and tested as positive among a population of patients identified as suffering from said disease using a reference test), as well as the specificity, which is the percentage of patients who do not suffer from the disease and who were tested as negative among a population of patients identified as not suffering from said disease using a reference test, have been determined for each of the screened neurodegenerative diseases that are diagnosed thanks to the test designed in the present invention. A test with 100% sensitivity will recognize all patients with the disease by testing them positive. A test with 100% specificity will exclude the disease from all healthy patients.

With regard to the Alzheimer's disease with a noradrenaline concentration [NA] comprised between 3000 pmol/l and 15000 pmol/l (AD1), the maximum sensitivity is 94.74% and the maximum specificity is 95.77%.

With regard to Alzheimer's disease with a noradrenaline concentration [NA] comprised between 100 pmol/l and 2700 pmol/l (AD2), the maximum sensitivity is 95.65% and the maximum specificity is 88.89%.

With regard to mental depression (PSY), the maximum sensitivity is 83.33% and the maximum specificity is 100.00%.

With regard to dementia with Lewy body (DLB), the maximum sensitivity is 100.00% and the maximum specificity is 98.80%.

With regard to fronto-temporal dementia (FTD), the maximum sensitivity is 70.0% and the maximum specificity is 93.75%.

With regard to vascular dementia (VD), the maximum sensitivity is 42.86% and the maximum specificity is 97.70%.

Finally, with regard to the control patients (see above for the definition of the control patients), the maximum sensitivity is 57.89% and the maximum specificity is 100.00%.

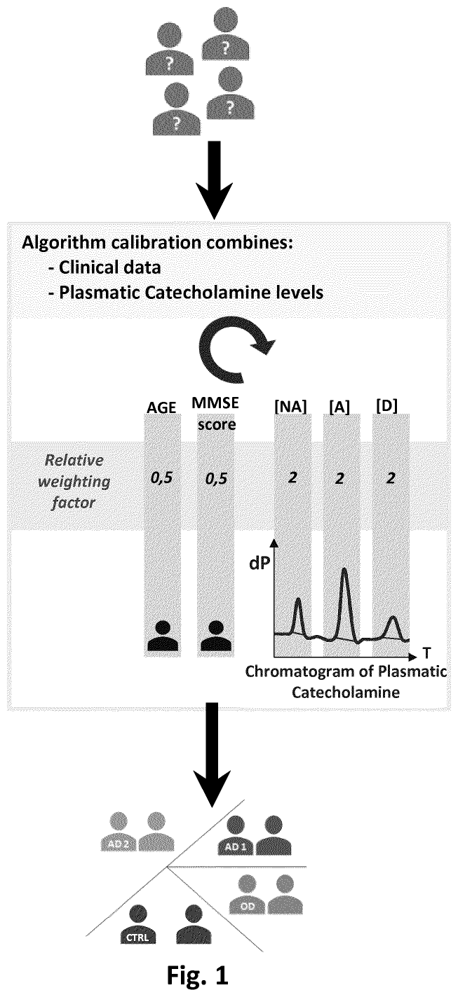

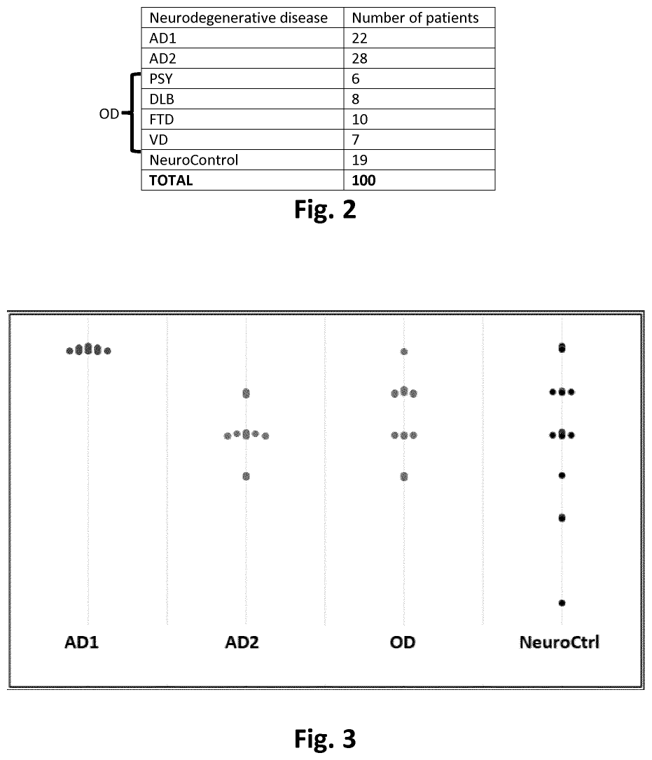

In order to achieve these highly significant statistical results, a reliable method of differential diagnosis of neurodegenerative disease has been performed. FIG. 1 conceptually shows the method of the present invention and FIG. 2 shows the panel of subjects from which the reference values have been determined. The abbreviation OD stands for “Other Diseases” and includes in fact PSY, DLB, FTD and VD. PSY (mental depression) also includes patients suffering from bipolar disorders.

The following criteria have been used in the present method:

The age of the patients that are to be diagnosed is collected, either by asking it clearly to the patients, or by asking it to one of his/her acquaintances, or by checking it in his/her identification papers.

Then, the patients are asked to respond to a medical questionnaire with regard to their cognitive performance. These tests consist of a set of 20 to 30 questions which assess basic cognitive functions. Most assessments require the individual to give basic personal information (i.e., name, address, etc.), to answer simple questions based on common knowledge (i.e., who is the president of the USA?), and to remember simple items of information such as a list of three words or name and address to recall later. According to the response given by the individual, a score is given that helps the clinician to categorize the patient. The questionnaires can be one of the abbreviated mental test (AMT), the mini-mental state examination (MMSE) and the six-item cognitive impairment test (6CIT). For the purpose of the present method of differential diagnosis, the MMSE has been chosen, but any other suitable medical questionnaire used to assess basic cognitive functions could be used.

Secondly, a blood sample of each individual is collected and is analysed to determine the level of three catecholamines (CA) into the plasma of the patients.

A simultaneous quantitation of epinephrine (adrenaline), norepinephrine (noradrenaline) and dopamine using a standardized HPLC method is performed. The HPLC is coupled with an electrochemical detection. The advantage of this screening is that the collection of blood sample is easy to perform on the individual, especially on the elderly, in comparison to the standard method of diagnosis of neurodegenerative diseases usually requiring the collection of cerebrospinal fluid. It is indeed known that a lumbar puncture is more invasive than blood collection and that lumbar puncture is not always feasible on all kind of patients.

The level of CA into the plasma of the individuals, the results of the MMSE as well as the age of the individuals are taken into account and processed in the following mathematical model.

These first five criteria (age, score to a medical questionnaire, adrenaline concentration ([A]), noradrenaline concentration ([NA]) and dopamine concentration ([D])) are now processed in order to determine which neurodegenerative disease the subject is suffering from.

A parameter based on the catecholamine concentrations can thus be determined in order to refine the diagnosis. It is indeed to be noted that the first five criteria are sufficient to provide the right diagnosis. In the case where a refinement is necessary, the experimentally-determined catecholamine concentrations will be process to determine the expected concentration of those catecholamines.

The determination of the expected concentration in the catecholamines is based on the measurement of the disparity between the measured concentration and a concentration of reference. The concentration of reference has been chosen to be the concentration of the catecholamines in the blood sample, more particularly the plasma, in patients suffering from Alzheimer's disease (AD). A mean value of such concentration has been determined among the initial panel of 100 subjects that has been selected in the training model step.

The adrenaline expected concentration corresponds to the following mathematical function:

[A]^{expected}=min(K₁;|[A]−[̅A̅]̅_̅{̅A̅l̅z̅h̅e̅i̅m̅e̅r̅}̅|) (1a)

meaning that the adrenaline expected concentration corresponds to the minimum value between K₁ and the modulus of the difference between the measured adrenaline concentration in the blood sample ([A]) with the mean adrenaline concentration in the blood sample for patients suffering from AD ([̅A̅]̅_̅{̅A̅l̅z̅h̅e̅i̅m̅e̅r̅}̅),

wherein

K₁ is a mathematical constant equal to 15. K₁ can be also comprised between 10 and 20 or between 5 and 25. In fact, K₁ can be all the integers between 5 and 25, and

[̅A̅]̅_̅{̅A̅l̅z̅h̅e̅i̅m̅e̅r̅}̅ is the average adrenaline concentration in the blood sample for patients suffering from AD. In fact, [A]_{Alzheimer} amounts to 483 pmol/l.

The noradrenaline expected concentration thus corresponds to the following mathematical function:

[NA]^{expected} min(K₁;|[NA]−[̅N̅A̅]̅_̅{̅A̅l̅z̅h̅e̅i̅m̅e̅r̅}̅|) (1b)

meaning that the noradrenaline expected concentration corresponds to the minimum value between K₁ and the modulus of the difference between the measured noradrenaline concentration in the blood sample ([NA]) with the average noradrenaline concentration in the blood sample for patients suffering from AD ([̅N̅A̅]̅_̅{̅A̅l̅z̅h̅e̅i̅m̅e̅r̅}̅),

wherein

K₁ is a mathematical constant equal to 15. K₁ can be also comprised between 10 and 20 or between 5 and 25. In fact, K₁ can be all the integers between 5 and 25, and

[̅N̅A̅]̅_̅{̅A̅l̅z̅h̅e̅i̅m̅e̅r̅}̅ is the average noradrenaline concentration in the blood sample for patients suffering from AD. In fact, [NA]_{Alzheimer} amounts to 4339 pmol/l.

The dopamine expected concentration thus corresponds to the following mathematical function:

[D]^{expected}=min(K₁;|[D]−[̅D̅]̅_̅{̅A̅l̅z̅h̅e̅i̅m̅e̅r̅}̅|) (1c)

meaning that the dopamine expected concentration corresponds to the minimum value between K₁ and the modulus of the difference between the measured dopamine concentration in the blood sample ([D]) with the average dopamine concentration in the blood sample for patients suffering from AD ([̅D̅]̅_̅{̅A̅l̅z̅h̅e̅i̅m̅e̅r̅}̅), wherein

K₁ is a mathematical constant equal to 15. K₁ can be also comprised between 10 and 20 or between 5 and 25. In fact, K₁ can be all the integers between 5 and 25, and

[̅D̅]̅_̅{̅A̅l̅z̅h̅e̅i̅m̅e̅r̅}̅ is the average dopamine concentration in the blood sample for patients suffering from AD. In fact, [̅D̅]̅_̅{̅A̅l̅z̅h̅e̅i̅m̅e̅r̅}̅ amounts to 454 pmol/l.

Those values obtained for one subject, either measured (for the age, the score, [A], [NA] and [D]) or determined ([A]^{expected}, [NA]^{expected}, and [D]^{expected}) are compared to a set of reference values. The eight criteria have been measured and/or determined and the following reference tables (Table I and Table II) have been established.

Table I shows the minimum reference values that have been determined in respect to the five criteria (age, score to the medical questionnaire, [NA], [A] and [D]) in relation with the neurodegenerative disease. The minimum for the three calculated criteria used in case of the refinement of the diagnosis method is also indicated ([NA]^{expected}, [A]^{expected}, and [D]^{expected}).

TABLE I
Reference table showing the lowest value of each criterion
in function of the neurodegenerative disease.

| | | MMSE | | | | | | |
|---|---|---|---|---|---|---|---|---|
| | Age | score | [NA] | [A] | [D] | [NA]^{expected} | [A]^{expected} | [D]^{expected} |
| AD1 | 57.8 | 20 | 2963 | 66 | 111 | 29 | 9 | 14 |
| AD2 | 51.9 | 4 | 1023 | 143 | 153 | 2 | 9 | 8 |
| PSY | 40.2 | 22 | 1534 | 191 | 276 | 19 | 21 | 13 |
| DLB | 61.7 | 8 | 1065 | 104 | 204 | 55 | 8 | 2 |
| FTD | 49.0 | 0 | 874 | 123 | 246 | 60 | 43 | 7 |
| VD | 58.8 | 21 | 1518 | 240 | 199 | 226 | 145 | 43 |
| Control | 40.2 | 22 | 925 | 164 | 208 | 27 | 71 | 22 |

Table II shows the maximum reference values that have been determined in respect to the five criteria (age, score to the medical questionnaire, [NA], [A] and [D]) in relation with the neurodegenerative disease. The maximum for the three calculated criteria used in case of the refinement of the diagnosis method is also indicated ([NA]^{expected}, [A]^{expected}, and [D]^{expected}).

TABLE II
Reference table showing the highest value of each criterion
in function of the neurodegenerative disease.

| | | MMSE | | | | | | |
|---|---|---|---|---|---|---|---|---|
| | Age | score | [NA] | [A] | [D] | [NA]^{expected} | [A]^{expected} | [D]^{expected} |
| AD1 | 87.7 | 29 | 8681 | 1721 | 878 | 4255 | 1228 | 415 |
| AD2 | 89.8 | 27 | 2539 | 1372 | 656 | 804 | 932 | 279 |
| PSY | 74.5 | 30 | 3691 | 407 | 519 | 1257 | 123 | 127 |
| DLB | 81.6 | 24 | 7217 | 418 | 375 | 4727 | 158 | 83 |
| FTD | 75.5 | 27 | 5744 | 1271 | 1003 | 3225 | 669 | 577 |
| VD | 85.7 | 29 | 3706 | 1526 | 533 | 1175 | 1032 | 230 |
| Control | 84.7 | 30 | 6331 | 2366 | 1334 | 3675 | 1788 | 838 |

Therefore, for subject suffering from Alzheimer's disease with a noradrenaline concentration [NA] comprised between 3000 pmol/l and 15000 pmol/l (AD1), the reference values for the criteria are as follows:
the age is comprised between 57.8 years and 87.7 years;
the score at the MMSE is comprised between 20 and 29;
[NA] is comprised between 2963 pmol/l and 8681 pmol/l;
[A] is comprised between 66 pmol/l and 1721 pmol/l;
[D] is comprised between 111 pmol/l and 878 pmol/l;
[NA]^{expected} is comprised between 29 pmol/l and 4255 pmol/l;
[A]^{expected} is comprised between 9 pmol/l and 1228 pmol/l;
[D]^{expected} is comprised between 14 pmol/l and 415 pmol/l.

Said reference values can be in fact written as being comprised between MINI_{AD1}^{criterion} and MAXI_{AD1}^{criterion}.

For subject suffering from Alzheimer's disease with a noradrenaline concentration [NA] comprised between 100 pmol/l and 2700 pmol/l (AD2), the reference values for the criteria are as follows:
the age is comprised between 51.9 years and 89.8 years;
the score at the MMSE is comprised between 4 and 27;
[NA] is comprised between 1023 pmol/l and 2539 pmol/l;
[A] is comprised between 143 pmol/l and 1372 pmol/l;
[D] is comprised between 153 pmol/l and 656 pmol/l;
[NA]^{expected} is comprised between 2 pmol/l and 804 pmol/l;
[A]^{expected} is comprised between 9 pmol/l and 932 pmol/l;
[D]^{expected} is comprised between 8 pmol/l and 279 pmol/l.

Said reference values can be in fact written as being comprised between MINI_{AD2}^{criterion} and MAXI_{AD2}^{criterion}.

For subject suffering from mental depression (PSY), the reference values for the criteria are as follows:
the age is comprised between 40.2 years and 74.5 years;
the score at the MMSE is comprised between 22 and 30.0;
[NA] is comprised between 1534 pmol/l and 3691 pmol/l;
[A] is comprised between 191 pmol/l and 407 pmol/l;
[D] is comprised between 276 pmol/l and 519 pmol/l;
[NA]^{expected} is comprised between 19 pmol/l and 1257 pmol/l;
[A]^{expected} is comprised between 21 pmol/l and 123 pmol/l;
[D]^{expected} is comprised between 13 pmol/l and 127 pmol/l.

Said reference values can be in fact written as being comprised between MINI_{PSY}^{criterion} and MAXI_{PSY}^{criterion}.

For subject suffering from dementia with Lewy body (DLB), the reference values for the criteria are as follows:
the age is comprised between 61.7 years and 81.6 years;
the score at the MMSE is comprised between 8 and 24;
[NA] is comprised between 1065 pmol/l and 7217 pmol/l;
[A] is comprised between 104 pmol/l and 418 pmol/l;
[D] is comprised between 204 pmol/l and 375 pmol/l;
[NA]^{expected} is comprised between 55 pmol/l and 4727 pmol/l;
[A]^{expected} is comprised between 8 pmol/l and 158 pmol/l;
[D]^{expected} is comprised between 2 pmol/l and 83 pmol/l.

Said reference values can be in fact written as being comprised between MINI_{DLB}^{criterion} and MAXI_{DLB}^{criterion}.

For subject suffering from fronto-temporal dementia (FTD), the reference values for the criteria are as follows:
the age is comprised between 49.0 years and 75.5 years;
the score at the MMSE is comprised between 0 and 27.0;
[NA] is comprised between 874 pmol/l and 5744 pmol/l;
[A] is comprised between 123 pmol/l and 1271 pmol/l;
[D] is comprised between 246 pmol/l and 1003 pmol/l;
[NA]^{expected} is comprised between 60 pmol/l and 3225 pmol/l;
[A]^{expected} is comprised between 43 pmol/l and 669 pmol/l;
[D]^{expected} is comprised between 7 pmol/l and 577 pmol/l.

Said reference values can be in fact written as being comprised between MINI_{FTD}^{criterion} and MAXI_{FTD}^{criterion}.

For subject suffering from vascular dementia (VD), the reference values for the criteria are as follows:
the age is comprised between 58.8 years and 85.7 years;
the score at the MMSE is comprised between 21 and 29;
[NA] is comprised between 1518 pmol/l and 3706 pmol/l;
[A] is comprised between 240 pmol/l and 1526 pmol/l;
[D] is comprised between 199 pmol/l and 533 pmol/l;
[NA]^{expected} is comprised between 226 pmol/l and 1175 pmol/l;
[A]^{expected} is comprised between 145 pmol/l and 1032 pmol/l;
[D]^{expected} is comprised between 43 pmol/l and 230 pmol/l.

Said reference values can be in fact written as being comprised between MINI_{VD}^{criterion} and MAXI_{VD}^{criterion}.

For a healthy control, the reference values for the criteria are as follows:
the age is comprised between 40.2 years and 84.7 years;
the score at the MMSE is comprised between 22 and 30;
[NA] is comprised between 925 pmol/l and 6331 pmol/l;
[A] is comprised between 164 pmol/l and 2366 pmol/l;
[D] is comprised between 208 pmol/l and 1334 pmol/l;
[NA]^{expected} is comprised between 27 pmol/l and 3675 pmol/l;
[A]^{expected} is comprised between 71 pmol/l and 1788 pmol/l;
[D]^{expected} is comprised between 22 pmol/l and 838 pmol/l.

Said reference values can be in fact written as being comprised between MINI_{healthy control}^{criterion} and MAXI_{healthy control}^{criterion}.

A correction factor δ can be attributed to these lowest and highest reference values. Said correction factor δ can be comprised between 0.50 and 1.50. Preferentially, said correction factor δ can be comprised between 0.75 and 1.25. If the correction factor δ is equal to 1, then the reference values that are used in the diagnostic method of the present invention are in fact the reference values as shown in Table I and in Table II.

The correction factor δ is used to take into consideration any possible deviation that can occur during the measurements of the catecholamine concentrations or the possible errors made by inadvertence at the determination of the MMSE score. In fact, for a correction factor δ different from 1, the values of Table I and Table II slightly differ. Iteration of the method of the present invention on one subject with different reference tables (obtained via different correction factor α) can provide a better robustness of the results. For instance, if a patient is diagnosed with fronto-temporal dementia with 3 different reference tables, then the certainty of the diagnosis is increased in comparison if said patient would have been diagnosed as suffering from this specific disease by reference to only 1 reference table.

The reference values, or the corrected reference values, are used to compare the at least five criteria (or even the eight criteria). The comparison step is in fact determinant to express a global note (or a global score) for each neurodegenerative disease. To obtain the global note, which will be used to diagnose the subject, a virtual value is firstly determined for each criterion.

If the subject presents a value for a criterion which is outside the range of the reference values (the lower limit being expressed in Table I, or in a corrected Table I if a correction factor δ has been applied; the upper limit being expressed in Table II, or in a corrected Table II in case where a correction factor δ is applied), then the virtual value is set to zero. In other words, the selected criterion does not count.

If the subject presents a value for a criterion which is inside the range of the reference values (as indicated above, in accordance with the reference values of (corrected) Table I and (corrected) Table II), then, the following mathematical equation is applied:

$${V}_{d i s e a s e}^{c r i t e r i o n} = 1 + K \frac{V A {L}^{criterion} - {MINI}_{dise ase}^{criterion}}{{MAXI}_{disease}^{criterion} - {MINI}_{dise ase}^{criterion}} \qquad ( 2 )$$

wherein VAL^{criterion} is the measured value corresponding to a criterion. For instance, if the virtual value for the criterion of age of the subject is to be determined, VAL represents the actual age of said subject. However, if the virtual value for the level of adrenaline in the blood sample of the subject is to be determined, then VAL is the actual measured level of adrenaline in the blood sample (or in the plasma) of the subject.

MINI_{disease}^{criterion} and MAXI_{disease}^{criterion} represent respectively the lowest and the highest reference values obtained, possibly adjusted with the correction factor δ. In fact, the lowest reference values (without applying the correction factor δ) are those listed in Table I and the highest reference values are those listed in Table II (see also paragraphs below said tables).

K is a mathematical parameter, varying between 0 and 0.1. Preferentially, K is comprised between 0 and 0.05.

In order to determine the global note in relation with each neurodegenerative disease, the virtual value in relation with all of the five criteria (age, score, [A], [NA], [D]) and/or with the three supplementary criteria (([A]^{expected}, [NA]^{expected}, and [D]^{expected}) is to be determined in accordance with the previous mathematical equation. Five or eight virtual values are thus calculated.

A weighting factor is then attributed to each of the virtual values. The virtual values in relation with the age and with the score are less relevant than the virtual values in relation with the concentration or the expected concentration. It is indeed more objective to determine scientifically a concentration of a catecholamine in the blood than asking questions to a person. The age is also a more subjective parameter since its relation with respect to the disease is rather variable and depends on the individuals rather than a determined law. For those reasons, the weighting factor for the virtual values in respect of the age and the score of the medical questionnaire has been set for instance to 0.5, while the weighting factor for the virtual values in respect with the concentrations or the expected concentration has been set for instance to 2.

The global note in relation with each neurodegenerative disease is merely obtained by adding all the weighted virtual values together. The mathematical writing is thus as follows:

GLOBAL NOTE=w₁V_{age}+w₁V_{score}+w₂(V_{[A]}+V_{[NA]}+V_{[D]})+w₂(V_{[A]}^{expected}+V_{[NA]}^{expected}+V_{[D]}^{expected}) (3)

wherein w₁ is a number comprised between 0.1 and 0.9, preferentially between 0.3 and 0.7. For instance, w₁ is equal to 0.5.

wherein w₂ is a number comprised between 1.1 and 3.0, preferentially between 1.5 and 2.5. For instance, w₂ is equal to 2.

The global note in relation with each neurodegenerative disease obtained for a sample of 13 subjects is indicated in Table III. To make the diagnosis, the clinician compares the values of the obtained global notes for each of the six neurodegenerative diseases and concludes that the patient suffers from a specific disease if the global note for this specific disease is the highest among the six global notes. The last three columns of Table III show the conclusion of the diagnosis, the 1^{st} diagnosis being the one which is the most probable for the patient. When the interval between the global notes is too weak, typically when the interval is inferior to 2.00 (preferentially inferior to 1.00, more preferentially inferior to 0.50), then a second diagnosis is made. Such patients, where two diagnosis can be made, or even when a third diagnosis can be made (also shown in Table III), generally suffers from mixed dementia. It has also been determined that patient suffering from mental depression (PSY) also shown signs of Alzheimer's disease (AD), mental depression being precursory signs of Alzheimer's disease (AD).

TABLE III
Global note in relation with each neurodegenerative disease calculated for 13 subjects
and conclusion of the differential diagnosis method of the present invention.

| | AD1 | AD2 | PSY | DLB | FTD | VD | Control | 1^{st} | 2^{nd} | 3^{rd} |
|---|---|---|---|---|---|---|---|---|---|---|
| 1 | 35.50 | 35.73 | 30.56 | 30.71 | 35.48 | 40.64 | 40.48 | VD | Control | |
| 2 | 30.46 | 35.76 | 41.45 | 20.52 | 40.65 | 30.76 | 40.44 | PSY | FTD | Control |
| 3 | 25.32 | 35.64 | 40.61 | 25.36 | 35.40 | 20.40 | 40.23 | PSY | Control | |
| 4 | 20.20 | 30.57 | 40.44 | 30.78 | 30.38 | 25.27 | 25.18 | PSY | | |
| 5 | 40.53 | 30.58 | 41.35 | 20.35 | 40.73 | 25.75 | 35.55 | PSY | DFT | AD1 |
| 6 | 30.32 | 35.66 | 41.08 | 30.55 | 35.44 | 25.47 | 35.53 | PSY | | |
| 7 | 30.46 | 35.57 | 40.79 | 35.62 | 30.32 | 35.57 | 35.44 | PSY | | |
| 8 | 30.36 | 30.55 | 30.76 | 40.86 | 30.62 | 35.46 | 35.36 | DLB | | |
| 9 | 30.36 | 30.50 | 10.05 | 40.91 | 30.35 | 15.28 | 25.36 | DLB | | |
| 10 | 30.32 | 40.77 | 20.48 | 41.10 | 30.63 | 35.34 | 35.32 | DLB | AD2 | |
| 11 | 25.20 | 40.56 | 35.73 | 40.72 | 40.39 | 25.27 | 30.26 | DLB | AD2 | FTD |
| 12 | 25.21 | 40.70 | 30.64 | 25.47 | 40.52 | 25.32 | 30.27 | AD2 | FTD | |
| 13 | 25.46 | 20.45 | 20.43 | 20.44 | 40.66 | 25.66 | 35.40 | FTD | | |

In fact, for each patient, the distance between the score of the patient and the cloud of patients in each category is measured. For instance, when in formula (2) the reference values for AD1 are taken into account (namely the value for MINI_{disease}^{criterion} and the value for MAXI_{disease}^{criterion} are those corresponding to MINI_{AD1}^{criterion} and to MAXI_{AD1}^{criterion} i.e., for example, in case where the criterion of [NA] is concerned, 2963 and 8681, respectively), one representation as the one depicted in FIG. 3 can be drawn. In this case, all the patients suffering from AD1 have high and concentrated score. In the other three categories (AD2, OD and Control), there are some subjects who also show scores similar to the scores of the patients in the AD category. But, this does not mean that they would eventually be diagnosed as suffering from AD, because they may have obtained a better score in another category.

That is the reason why the global note must be calculated by taking into account all the neurodegenerative diseases under study. For instance, when the reference values in formula (2) are used for the control group, a representation as the one shown in FIG. 4 is drawn for the very same group of patient.

By applying this algorithm to each patient, the final suggested diagnosis is the diagnosis of the cloud of points (amongst the 8 possible original clouds) to which the patient is the closest according to the distance measured. If a second-best diagnosis (as shown in Table III) (and possibly a third-best diagnosis) has a close enough score, this second-best, and potentially the third-best, may also be indicated to the clinician.

It is therefore possible for an individual to be diagnosed from a neurodegenerative disease or from mixed dementia. The attribution of a global note for each neurodegenerative disease and for each patient allows the clinician not only to categorize the patient in one class of disease (for instance, by assessing that said patient suffers from AD) but also to give an insight to what other disease this same patient could suffer from (based on the second-best and potentially the third-best diagnosis).

An additional criterion that is used is the determination whether the subject in question suffers from arterial hypertension. It has been indeed noticed that the prevalence of arterial hypertension is four times less in patients suffering from AD. This could be linked to the formation of aggregate in the brain and the low clearance of those aggregates (due to (extremely) low blood pressure in these patients). Therefore, the determination of the blood pressure in the subject can be a further parameter in order to determine whether the subject suffers from AD.

The accurate detection and identification of catecholamines simultaneously in the blood lays a solid foundation for the study of the potential links between AD and catecholamines. In this exploratory study, with a cohort of patients (n=100) suffering from dementia and other neurodegenerative diseases, we demonstrate the interest of the plasma catecholamine assay for the development of less invasive and accessible solution for the diagnosis of Alzheimer's disease (AD1+AD2), other neurodegenerative diseases, such as PSY, DLB, FTD, VD and also mixed dementia. The statistical exploitation of the signature of catecholamines, coupled with simple data such as age, the mini-mental state examination (MMSE) and the determination of blood pressure, makes it possible to reach for the diagnosis of subject suffering from Alzheimer's disease with a noradrenaline concentration [NA] comprised between 3000 pmol/l and 15000 pmol/l (AD1) with high sensitivity and specificity performances, namely with 94.74% and 95.77% respectively and also for the diagnosis of subject suffering from Alzheimer's disease with a noradrenaline concentration [NA] comprised between 100 pmol/l and 2700 pmol/l (AD2) (95.65% and 88.89% of sensitivity and specificity respectively).

The diagnosis method of the present invention is very useful not only to diagnose a subject, but it is also a tool for providing the follow-up of those subjects. In particular, it is highlighted that the preparation of samples is performed easily, since it is based on a simple blood test. For the medical research point of view, this diagnosis method is also very practical, since, due to the simple detection method used, new treatment and new medicines can be easily assessed.

Analytical Method

Blood collected in lithium heparin tubes was centrifuged at 3500 rpm for 10 min at +4° C. 1 mL plasma and 100 μL of 2,3-dihydroxybenzoic acid (DHBA) internal standard (from 100 nM DHBA stock solution) was added to extraction tubes with 100 mg of aluminum oxide previously activated with a mixture of 500 μL TRIS 3M and 100 μL EDTA 10% (5:1) according to a known method (Anton A. H., et al., J. Pharmacol. Exp. Ther., 1962, 138(3), 360-375). Samples were stirred for 10 min and centrifuged at 2000 rpm at 4° C. The aluminum oxide with bound catecholamines was then washed 3 times with 5 mL of distilled water followed by centrifugation. The elution of catecholamines was achieved by the addition of 500 μL of HClO₄ 0.2 N and subsequent centrifugation at 2000 rpm for 10 min at 4° C. Aliquots of 100 μL were injected into the HPLC system (Waters 515 HPLC Pump, Waters Model 717 autosampler injector), which was equipped with a Purospher® STAR RP-C18 endcapped (5 μm). The mobile phase consisted of 50 mM sodium acetate buffer containing 0.9 mM sodium lauryl sulfate, 0.3 mM EDTA, 17.5 mM acetic acid and 12% methanol (vol/vol) at pH 3.6. Electrochemical detection (Coulochem II detector) was performed using an ESA 5010 cell with a glassy carbon working electrode set at a potential of +360 mV vs. Ag/AgCl.

Description of Sample Preparation

The catecholamines (adrenaline, noradrenaline and dopamine) are extracted from the plasma matrix by adsorption on alumina before the HPLC analysis.

Sample preparation is simple, because pH-adjustment of the plasma samples is not necessary. Moreover, samples preparation requires only washing steps with Wash Buffer. A selected HPLC column in combination with mobile phase, optimised for this particular separation, allows for sure and reliable chromatographic quantification. With a certified HPLC kit, one person can analyse up to 100 plasma samples per day.

Extraction: Label a sample clean up cartridge appropriately for each sample. Add 0.5 ml extraction buffer to each cartridge and shake briefly. Then add 1 ml plasma (total capacity of the cartridge is 1.5 ml) and 50 μl Internal Standard (=600 pg DHBA). Close the cartridge with the top plug and mix for 10 min. Then remove the bottom plug from the cartridge and remove the plasma supernatant by using a vacuum equipment or centrifugation (place the cartridge in a disposable centrifugation tube and centrifuge 1 min at 2000 rpm).

Wash steps: Re-mount the bottom plug and remove the top plug. Add 1 ml Wash Buffer. Close the cartridge again and mix for 30 s (vortex). Then remove the bottom plug from the cartridge and remove the plasma supernatant by using a vacuum equipment or centrifugation (place the cartridge in a disposable centrifugation tube and centrifuge 1 min at 2000 rpm). Repeat this step 2 more times. After the last (third) wash step dry the cartridges well by centrifugation (2 min at 4000 rpm). To ensure that the Wash Buffer is removed completely, tap at the cartridge; the alumina should loosen from the frit. Discard bottom plug.

Elution: Prior to elution attach the plastic tube supplied with the kit to the outlet of the sample clean up column. Add 120 μl Elution Buffer, shake briefly, and wait for 5 min. Then mix for 30 s (vortex). Place the sample clean up column (with the plastic tube attached) in a fresh vial and centrifuge 1 min at 2000 rpm. The eluted sample is collected in the attached tube (This tube can be directly placed into a Waters WISP autosampler).

Low Concentration of Noradrenaline

A low concentration of noradrenaline is significative of a hypo-activity of the brain, specifically a low production of noradrenaline in the locus coeruleus of the brain. This nucleus in the brainstem is responsible for the brain synthesis of noradrenaline. This noradrenaline can in fact be released into the blood system through the adrenal gland. The hypo-activity of the brain, generally correlated with low score to the medical questionnaire for screening cognitive function, for instance with a low MMSE score, namely inferior to 15, may be an indication that the patient suffers from an advance stage of Alzheimer's disease (AD2).

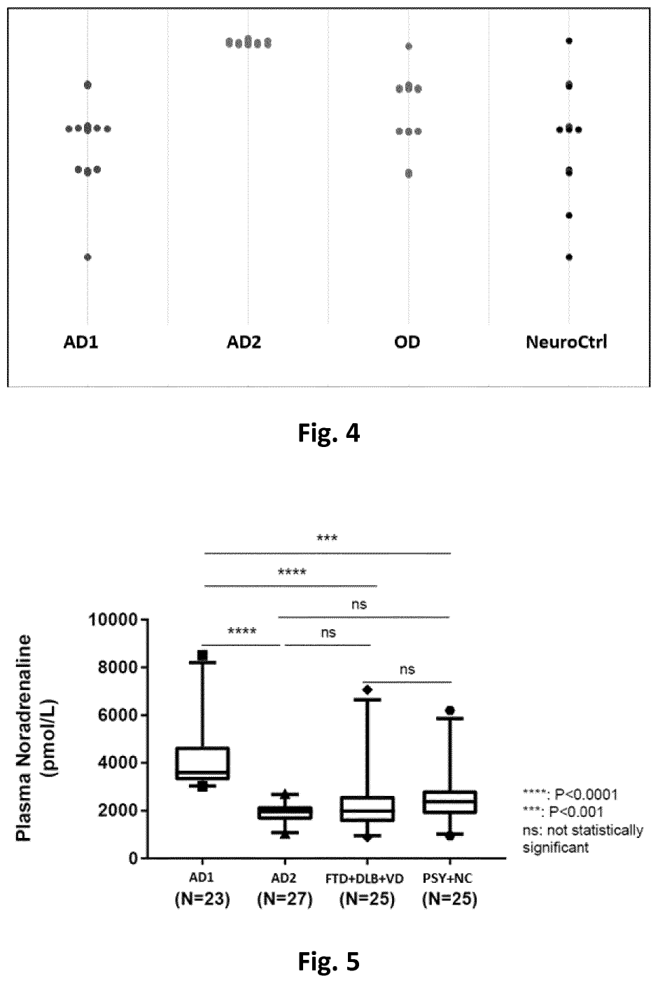

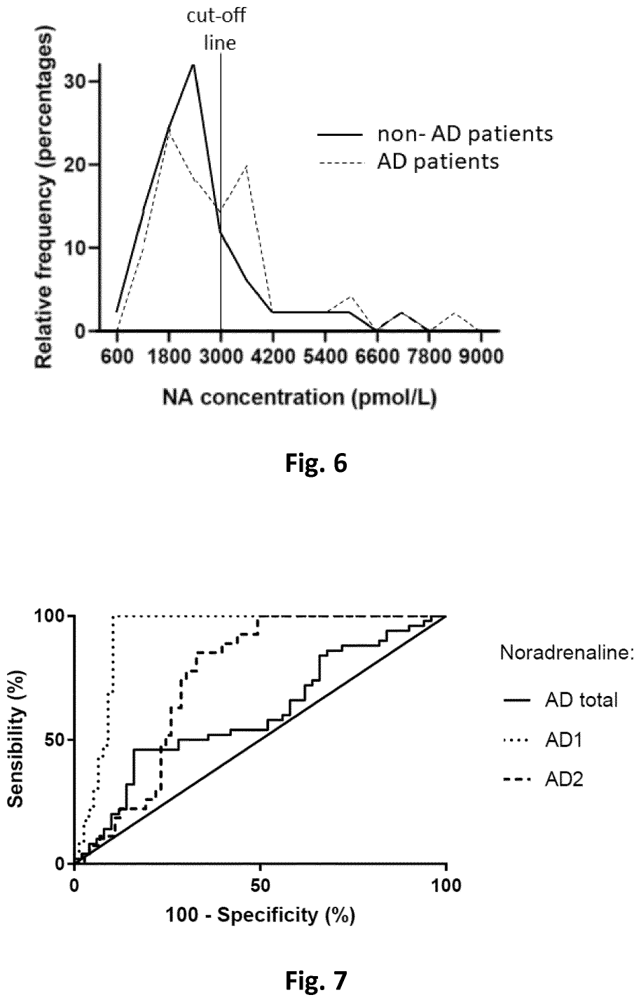

Statistical studies, based notably on Receiver Operating Characteristic (ROC) curves, have allowed to determine a threshold with respect to the noradrenaline concentration in order to classify the subject as potentially suffering from AD1 or from AD2. ROC curves are graphical plot that illustrates the diagnosis ability of a binary classifier system as its discrimination threshold is varied. Indeed, the plasma noradrenaline data sample was not normally distributed and the histogram distribution of the samples did not show two groups of samples equally distributed (see FIG. 5). The relative frequency distribution of plasma noradrenaline concentration of AD patient versus non-AD patient is a visual way to determine a threshold with respect to the noradrenaline concentration in order to classify the subject as potentially suffering from AD1 or from AD2 (see FIG. 6). Indeed, it can be clearly seen that among all the patients with similar noradrenaline concentration, the AD patients are distributed into two distinct distribution, with a cut-off line comprised between 2900 pmol/l and 3100 pmol/l. The ROC analysis (see FIG. 7) has also allowed to identify the optimal cut-off that maximizes the Youden Index for the noradrenaline. The area under the curve (AUC) is not very good because the noradrenaline alone is not enough specific to distinguish between AD patients and non-AD patient. However, this cut-off, when applied to separate two sub-types of AD patients (AD1 and AD2), provides a very good discrimination (especially for AD1, since the AUC is the greatest among the one of AD1 and the one of AD2). The ROC curve analysis of plasma NA concentrations in AD versus non-AD patients has allowed to identify 3023 pmol/l as the optimal cut-off that maximises the Youden Index.

It is thus safe to affirm that when a subject has a noradrenaline concentration [NA] lower or equal to a predetermined threshold that is comprised between 2900 pmol/l and 3100 pmol/l, then the subject is suspected to suffer from an advanced stage of Alzheimer's disease (AD2). For practical feasibility, it is preferred that the cut-off for distinguishing between AD1 and AD2 is set to a noradrenaline concentration of at least 3000 pmol/l.

The suspicion is however not sufficient to base an accurate diagnosis on one patient. Further investigation is therefore needed to established an accurate and precise diagnosis.

Arterial Hypertension

Once the concentration in noradrenaline has been determined in a subject, and in the case where this concentration is inferior or equal to the predetermined threshold, preferably inferior or equal to 3000 pmol/l, the arterial hypertension status (also referenced as the high blood pressure status, or HBP status) of the patient is determined.

To do so, it is checked whether the subject has blood pressure of at least 120/80 mmHg for at least three consecutive days. The first number corresponds to the systolic pressure and the second number corresponds to the diastolic pressure. Preferably, in order to be determined as suffering from arterial hypertension, the subject should present a blood pressure of at least 140/90 mmHg for at least three consecutive days. By comparison, the normal blood systolic pressure of a healthy patient is ranging between 90 and 119 mmHg while the normal blood diastolic pressure of a healthy patient is ranging between 60 and 79 mmHg.

In the case where the blood pressure is inferior to 140/90 mmHg during at least 3 consecutive days, preferentially inferior to 120/80 mmHg during at least 3 consecutive days, it can be concluded that the subject does not suffer from arterial hypertension.

Determination of a Global Note for Each Neurodegenerative Disease

The experimental data (age, score, concentrations) are then processed with a mathematical algorithm in order to determine a global note which will be used to assess the final diagnosis. Experimental data such as sex and presence of the APOEe4 allele in the genotype can also be included. However, they can also be omitted. The determination of the global note first requires the determination of parameters.

As the subject to be diagnosed is, before the diagnosis, not categorized as suffering from one specific neurodegenerative disease in particular, but rather categorized as suffering from one of the medical conditions selected among six diseases (AD1, AD2, PSY, DLB, FTD, VD) in addition to the neurological control (NC), the mathematical algorithm has been derivatized in a way that each of the experimental clinical data (age, MMSE score, [A], [NA], [D] and extent of arterial hypertension in the case where [NA]>3000 pmol/l) is transformed into one parameter in function of one of the seven medical conditions that are studied in the present case.

That is the reason why for one subject presenting a [NA] superior to 3000 pmol/l, 35 parameters are determined. There is indeed no checking of the arterial hypertension of said subject.

However, when a subject has a [NA] inferior to 3000 pmol/l, the arterial hypertension is determined and therefore, there are a total of 42 parameters to be considered.

When the parameters are determined, they can be processed into a global note in relation with each of the neurological diseases under study. The global note thus computed will serve as basis to conclude on a final diagnosis for said subject.

In equation (2) K is a mathematical parameter that can be comprised between 0.01 and 1.00, preferentially between 0.02 and 0.99, more preferentially between 0.05 and 0.95.

In a preferred embodiment; the reference values in relation with early stage of Alzheimer's disease (AD1) and implemented in equation (2) are as followed:
with respect to the age of said subject, the minimum reference value is at least 50 years and/or at most 60 years, preferably at least 51 year, more preferably at least 52 year and most preferably is 52.92; and/or the maximum reference value is at least 85 years and/or at most 90 years, preferably at most 89 years, more preferably at most 88 years, and most preferably is 87.72 years;
with respect to the score of said subject to a questionnaire adapted for screening cognitive function, the minimum reference value is at least 14 and/or at most 16, preferably is 15; and/or the maximum reference value is at least 27 and/or at most 29, preferably is 28;
with respect to the dopamine concentration [D], the minimum reference value is at least 90 pmol/l and/or at most 150 pmol/l, preferably at least 100 pmol/l, more preferably at least 105 pmol/l, and most preferably is 110.74 pmol/l; and/or the maximum reference value is at least 800 pmol/l and/or at most 1000 pmol/l, preferably at most 950 pmol/l, more preferably at most 925 pmol/l and most preferably is 878.22 pmol/l;
with respect to the adrenaline concentration [A] the minimum reference value is at least 40 pmol/l and/or at most 100 pmol/l, preferably at least 50 pmol/l, more preferably at least 60 pmol/l, and most preferably is 65.66 pmol/l; and/or the maximum reference value is at least 1500 pmol/l and/or at most 2000 pmol/l, preferably at most 1900 pmol/l, more preferably at most 1800 pmol/l and most preferably is 1720.74 pmol/l;
with respect to the noradrenaline concentration [NA] the minimum reference value is at least 2900 pmol/l and/or at most 3200 pmol/l, preferably at least 2950 pmol/l, more preferably at least 2960 pmol/l, and most preferably is 2962.54 pmol/l; and/or the maximum reference value is at least 8100 pmol/l and/or at most 9000 pmol/l, preferably at most 8800 pmol/l, more preferably at most 8700 pmol/l and most preferably is 8681.22 pmol/l.

In a preferred embodiment, the reference values used in relation with advanced stage of Alzheimer's disease (AD2) and implemented in equation (2) are as followed:
with respect to the age of said subject, the minimum reference value is at least 45 years and/or at most 55 years, preferably at least 47 year, more preferably at least 50 year and most preferably is 51.94; and/or the maximum reference value is at least 85 years and/or at most 95 years, preferably at most 92 years, more preferably at most 90 years, and most preferably is 89.76 years;
with respect to the score of said subject to a questionnaire adapted for screening cognitive function, the minimum reference value is at least 3 and/or at most 5, preferably is 4; and/or the maximum reference value is at least 28 and/or at most 30, preferably is 29;
with respect to the dopamine concentration [D], the minimum reference value is at least 140 pmol/l and/or at most 175 pmol/l, preferably at least 145 pmol/l, more preferably at least 150 pmol/l, and most preferably is 152.88 pmol/l; and/or the maximum reference value is at least 500 pmol/l and/or at most 1000 pmol/l, preferably at most 800 pmol/l, more preferably at most 700 pmol/l and most preferably is 655.86 pmol/l;
with respect to the adrenaline concentration [A] the minimum reference value is at least 80 pmol/l and/or at most 200 pmol/l, preferably at least 120 pmol/l, more preferably at least 130 pmol/l, and most preferably is 143.08 pmol/l; and/or the maximum reference value is at least 1100 pmol/l and/or most 1500 pmol/l, preferably at most 1450 pmol/l, more preferably at most 1400 pmol/l and most preferably is 1371.09 pmol/l;
with respect to the noradrenaline concentration [NA] the minimum reference value is at least 750 pmol/l and/or at most 1150 pmol/l, preferably at least 850 pmol/l, more preferably at least 1000 pmol/l, and most preferably is 1023.12 pmol/l; and/or the maximum reference value is at least 2600 pmol/l and/or at most 3000 pmol/l, preferably at most 2900 pmol/l, more preferably at most 2800 pmol/l and most preferably is 2758.08 pmol/l.

In a preferred embodiment, the reference values used in relation with mental depression (PSY) and implemented in equation (2) are as followed:
with respect to the age of said subject, the minimum reference value is at least 35 years and/or at most 45 years, preferably at least 37 year, more preferably at least 40 year and most preferably is 40.18; and/or the maximum reference value is at least 70 and/or at most 80 years, preferably at most 78 years, more preferably at most 76 years, and most preferably is 74.46 years;
with respect to the score of said subject to a questionnaire adapted for screening cognitive function, the minimum reference value is at least 21 and/or at most 23, preferably is 22; and/or the maximum reference value is at least 28 and/or at most 30, preferably is 29;
with respect to the dopamine concentration [D], the minimum reference value is at least 250 pmol/l and/or at most 320 pmol/l; preferably at least 260 pmol/l, more preferably at least 270 pmol/l, and most preferably is 276.36 pmol/l; and/or the maximum reference value is at least 450 pmol/l and/or at most 750 pmol/l, preferably at most 700 pmol/l, more preferably at most 600 pmol/l and most preferably is 519.18 pmol/l;
with respect to the adrenaline concentration [A] the minimum reference value is at least 180 pmol/l and/or at most 220 pmol/l; preferably at least 185 pmol/l, more preferably at least 190 pmol/l, and most preferably is 191.10 pmol/l; and/or the maximum reference value is at least 360 pmol/l and/or at most 450 pmol/l, preferably at most 420 pmol/l, more preferably at most 410 pmol/l and most preferably is 406.98 pmol/l;
with respect to the noradrenaline concentration [NA] the minimum reference value is at least 1000 pmol/l and/or at most 2000 pmol/l; preferably at least 1200 pmol/l, more preferably at least 1400 pmol/l, and most preferably is 1533.70 pmol/l; and/or the maximum reference value is at least 3000 pmol/l and/or at most 4000 pmol/l, preferably at most 3800 pmol/l, more preferably at most 3700 pmol/l and most preferably is 3691.38 pmol/l.

In a preferred embodiment, the reference values used in relation with dementia—with Lewy bodies (DLB) and implemented in equation (2) are as followed:
with respect to the age of said subject, the minimum reference value is at least 55 years and/or at most 65, preferably at least 57 year, more preferably at least 60 year and most preferably is 61.74; and/or the maximum reference value is at least 75 years and/or at most 85 years, preferably at most 83 years, more preferably at most 82 years, and most preferably is 81.6 years;
with respect to the score of said subject to a questionnaire adapted for screening cognitive function, the minimum reference value is at least 7 and/or at most 9, preferably is 8; and/or the maximum reference value is at least 23 and/or most 25, preferably is 24;
with respect to the dopamine concentration [D], the minimum reference value is at least 150 pmol/l and/or at most 250 pmol/l; preferably at least 170 pmol/l, more preferably at least 180 pmol/l, and most preferably is 203.84 pmol/l; and/or the maximum reference value is at least 300 pmol/l and/or at most 400 pmol/l, preferably at most 390 pmol/l, more preferably at most 380 pmol/l and most preferably is 375.36 pmol/l;
with respect to the adrenaline concentration [A] the minimum reference value is at least 80 pmol/l and/or at most 125 pmol/l; preferably at least 90 pmol/l, more preferably at least 100 pmol/l, and most preferably is 108 pmol/l; and/or the maximum reference value is at least 580 pmol/l and/or at most 720 pmol/l, preferably at most 690 pmol/l, more preferably at most 670 pmol/l and most preferably is 650 pmol/l;
with respect to the noradrenaline concentration [NA] the minimum reference value is at least 900 pmol/l and/or at most 1300 pmol/l; preferably at least 1000 pmol/l, more preferably at least 1050 pmol/l, and most preferably is 1065.26 pmol/l; and/or the maximum reference value is at least 6500 pmol/l and/or at most 8000 pmol/l, preferably at most 7500 pmol/l, more preferably at most 7300 pmol/l and most preferably is 7216.50 pmol/l.

In a preferred embodiment, the reference values used in relation with frontotemporal dementia (FTD) and implemented in equation (2) are as followed:
with respect to the age of said subject, the minimum reference value is at least 45 years and/or at most 53, preferably at least 47 year, more preferably at least 48 year and most preferably is 49; and/or the maximum reference value is at least 70 years and/or at most 80 years, preferably at most 78 years, more preferably at most 76 years, and most preferably is 75.48 years;
with respect to the score of said subject to a questionnaire adapted for screening cognitive function, the minimum reference value is at least 0 and/or at most 1, preferably is 0; and/or the maximum reference value is at least 26 and/or most 28, preferably is 27;
with respect to the dopamine concentration [D], the minimum reference value is at least 200 pmol/l and/or at most 400 pmol/l; preferably at least 230 pmol/l, more preferably at least 240 pmol/l, and most preferably is 245.98 pmol/l; and/or the maximum reference value is at least 900 pmol/l and/or at most 1100 pmol/l, preferably at most 1050 pmol/l, more preferably at most 1020 pmol/l, and most preferably is 1002.66 pmol/l;
with respect to the adrenaline concentration [A] the minimum reference value is at least 100 pmol/l and/or at most 150 pmol/l; preferably at least 110 pmol/l, more preferably at least 120 pmol/l, and most preferably is 123.48 pmol/l; and/or the maximum reference value is at least 1100 pmol/l and/or at most 1500 pmol/l, preferably at most 1400 pmol/l, more preferably at most 1300 pmol/l and most preferably is 1270.90 pmol/l;
with respect to the noradrenaline concentration [NA] the minimum reference value is at least 700 pmol/l and/or at most 1000 pmol/l; preferably at least 800 pmol/l, more preferably at least 850 pmol/l, and most preferably is 874.16 pmol/l; and/or the maximum reference value is at least 5300 pmol/l and/or at most 6000 pmol/l, preferably at most 5900 pmol/l, more preferably at most 5800 pmol/l and most preferably is 5743.62 pmol/l.

In a preferred embodiment, the reference values used in relation with vascular dementia (VD) and implemented in equation (2) are as followed:
with respect to the age of said subject, the minimum reference value is at least 55 years and/or at most 63, preferably at least 56 year, more preferably at least 58 year and most preferably is 58.8; and/or the maximum reference value is at least 80 years and/or at most 90 years, preferably at most 88 years, more preferably at most 86 years, and most preferably is 85.68 years;
with respect to the score of said subject to a questionnaire adapted for screening cognitive function, the minimum reference value is at least 20 and/or at most 22, preferably is 21; and/or the maximum reference value is at least 27 and/or most 29, preferably is 28;
with respect to the dopamine concentration [D], the minimum reference value is at least 140 pmol/l and/or at most 250 pmol/l; preferably at least 160 pmol/l, more preferably at least 180 pmol/l, and most preferably is 198.94 pmol/l; and/or the maximum reference value is at least 450 pmol/l and/or at most 580 pmol/l, preferably at most 560 pmol/l, more preferably at most 540 pmol/l, and most preferably is 533.46 pmol/l;
with respect to the adrenaline concentration [A] the minimum reference value is at least 200 pmol/l and/or at most 270 pmol/l; preferably at least 210 pmol/l, more preferably at least 230 pmol/l, and most preferably is 240.10 pmol/l; and/or the maximum reference value is at least 1200 pmol/l and/or at most 1800 pmol/l, preferably at most 1700 pmol/l, more preferably at most 1600 pmol/l and most preferably is 1525.92 pmol/l;
with respect to the noradrenaline concentration [NA] the minimum reference value is at least 1400 pmol/l and/or at most 1800 pmol/l; preferably at least 1450 pmol/l, more preferably at least 1500 pmol/l, and most preferably is 1518.02 pmol/l; and/or the maximum reference value is at least 3400 pmol/l and/or at most 4000 pmol/l, preferably at most 3900 pmol/l, more preferably at most 3800 pmol/l and most preferably is 3705.66 pmol/l.

In a preferred embodiment, the method further comprises calculating one global note in relation with neurological control (NC); with preference, the reference values used in step (c) in relation with neurological control (NC) and implemented in equation (2) are as followed:
with respect to the age of said subject, the minimum reference value is at least 35 years and/or at most 45, preferably at least 37 year, more preferably at least 39 year and most preferably is 40.18; and/or the maximum reference value is at least 80 years and/or at most 90 years, preferably at most 88 years, more preferably at most 86 years, and most preferably is 84.66 years;
with respect to the score of said subject to a questionnaire adapted for screening cognitive function, the minimum reference value is at least 22 and/or at most 24, preferably is 23; and/or the maximum reference value is at least 29 and/or most 30, preferably is 30;
with respect to the dopamine concentration [D], the minimum reference value is at least 180 pmol/l and/or at most 240 pmol/l; preferably at least 190 pmol/l, more preferably at least 200 pmol/l, and most preferably is 207.76 pmol/l; and/or the maximum reference value is at least 1250 pmol/l and/or at most 1600 pmol/l, preferably at most 1500 pmol/l, more preferably at most 1400 pmol/l, and most preferably is 1334.16 pmol/l;
with respect to the adrenaline concentration [A] the minimum reference value is at least 110 pmol/l and/or at most 200 pmol/l; preferably at least 130 pmol/l, more preferably at least 150 pmol/l, and most preferably is 163.66 pmol/l; and/or the maximum reference value is at least 2250 pmol/l and/or at most 2500 pmol/l, preferably at most 2450 pmol/l, more preferably at most 2400 pmol/l and most preferably is 2366.40 pmol/l;
with respect to the noradrenaline concentration [NA] the minimum reference value is at least 900 pmol/l and/or at most 1650 pmol/l; preferably at least 910 pmol/l, more preferably at least 920 pmol/l, and most preferably is 925.12 pmol/l; and/or the maximum reference value is at least 6000 pmol/l and/or at most 7000 pmol/l, preferably at most 6600 pmol/l, more preferably at most 6400 pmol/l and most preferably is 6331.14 pmol/l.

1.-22. (canceled)

23. An in vitro method for performing a differential diagnosis of neurodegenerative diseases in one subject, said subject being selected among subjects suffering from early stage of Alzheimer's disease AD1, advanced stage of Alzheimer's disease AD2, mental depression PSY, dementia with Lewy Body DLB, frontotemporal dementia FTD, and/or vascular dementia VD, said method comprising:
(a) determining a value VAL^{criterion} of at least five criteria of said subject, selected from the list
an age of said subject,
a score of said subject to a questionnaire adapted for screening cognitive function, preferentially a mini-mental state examination questionnaire,
a dopamine concentration [D] in one blood sample of said subject,
an adrenaline concentration [A] in one blood sample of said subject, and
a noradrenaline concentration [NA] in one blood sample of said subject;
(b) comparing said at least five criteria of said subject with reference values by calculating a global note in relation with each neurodegenerative disease, comprising the following sub-steps:
determining a virtual value V_{disease}^{criterion} by neurodegenerative disease in function of each criterion,
attributing a weighting factor to each virtual value V_{disease}^{criterion} determined in the previous sub-step,
determining a sum of each of said weighted virtual values so as to obtain the global note in relation with each neurodegenerative disease; and
(c) determining whether said subject suffers from AD1, AD2, PSY, DLB, FTD or VD based on the global note that is the highest.

24. The method according to claim 23, wherein, in step (b), determining a virtual value V_{disease}^{criterion} by neurodegenerative disease in function of each criterion comprises:
comparing the value VAL^{criterion} of the criterion with a range of the reference values delimited by a lower limit and an upper limit, where if said value is outside said range, said virtual value V_{disease}^{criterion} is zero and if said value VAL, VAL^{criterion} is inside said range said virtual value V_{disease}^{criterion} is positive and maximum when the value VAL^{criterion} is equal to the upper limit.

25. The method according to claim 23, wherein, in step (b), the virtual value V_{disease}^{criterion} by neurodegenerative disease in function of each criterion is zero if the value VAL^{criterion} of the criterion is outside a range of the reference values delimited by a lower limit MINI w and an upper limit MAXI_{disease}^{criterion} both for the neurodegenerative disease and the criterion, otherwise
$${V}_{d i s e a s e}^{c r i t e r i o n} = 1 + K \frac{V A {L}^{criterion} - {MINI}_{dise ase}^{criterion}}{{MAXI}_{disease}^{criterion} - {MINI}_{dise ase}^{criterion}}$$
wherein K is a mathematical parameter, K being preferentially comprised between 0 and 0.1.

26. The method according to claim 23, wherein the reference values used in step (b) are as followed:
the age of said subject is comprised within the range 40.2 years and 89.8 years,
the score of said questionnaire adapted for screening cognitive function is comprised between 0 and 30,
the dopamine concentration is comprised between 111 pmol/l and 1334 pmol/l,
the adrenaline concentration is comprised between 66 pmol/l and 2366 pmol/l,
the noradrenaline concentration is comprised between 874 pmol/l and 8681 pmol/l,
the dopamine expected concentration is comprised between 2 pmol/l and 838 pmol/l,
the adrenaline expected concentration is comprised between 8 pmol/l and 1788 pmol/l,
the noradrenaline expected concentration is comprised between 2 pmol/l and 4727 pmol/l.

27. The method according to claim 23, wherein the list of criteria further comprises:
a dopamine expected concentration [D]^{expected} in one blood sample of said subject,
an adrenaline expected concentration [A]^{expected} in one blood sample of said subject,
a noradrenaline expected concentration [NA]^{expected} in one blood sample of said subject.

28. The method according to claim 27, wherein said step (a) is a step of determining at least eight criteria.

29. The method according claim 27, wherein said dopamine expected concentration is obtained by applying the following equation
[D]^{expected}=min(K₁;|[D]−[̅D̅]̅_̅{̅A̅l̅z̅h̅e̅i̅m̅e̅r̅}̅|)
wherein K₁ is a constant comprised between 5 and 25, and
wherein [D]_{Alzheimer} is 454 pmol/l.

30. The method according to claim 27, wherein said adrenaline expected concentration is obtained by applying the following equation
[A]^{expected}=min(K₁;|[A]−[̅A̅]̅_̅{̅A̅l̅z̅h̅e̅i̅m̅e̅r̅}̅|)
wherein K₁ is a constant comprised between 5 and 25, and
wherein [A]_{Alzheimer} is 483 pmol/l.

31. The method according to claim 27, wherein said noradrenaline expected concentration is obtained by applying the following equation
[NA]^{expected}=min(K₁;|[NA]−[̅N̅A̅]̅_̅{̅A̅l̅z̅h̅e̅i̅m̅e̅r̅}̅|)
wherein K₁ is a constant comprised between 5 and 25, and
wherein [NA]_{Alzheimer} is 4339 pmol/l.

32. The method according to claim 23, further comprising:
the step of attributing a correction factor δ to said reference values, said correction factor δ being comprised between 0.50 and 1.50.

33. The method according to claim 23, wherein the weighting factor (w₁) in relation with the virtual value V_{disease}^{criterion} with respect to the age of said subject and to the score of said subject is comprised between 0.1 and 0.9, preferentially is equal to 0.5 and wherein the weighting factor (w₂) in relation with the virtual value V_{disease}^{criterion} with respect to the concentrations and the expected concentrations comprised between 1.1 and 3.0, preferentially is equal to 2.

34. The method according to claim 23, wherein said method further comprises a step (a′) performed after step (a); wherein step (a′) comprises the step of comparing said noradrenaline concentration [NA] to a predetermined threshold, and if said noradrenaline concentration [NA] is lower or equal to said predetermined threshold, then determining the arterial hypertension status of said subject, and in that step (c) further comprises, if said noradrenaline concentration [NA] is lower or equal to said predetermined threshold, the step of calculating the global note in relation with AD2 by adding a parameter in relation with the arterial hypertension status of said subject.

35. The method according to claim 34, characterized in that the predetermined threshold of step (a′) is a noradrenaline concentration [NA] comprised between 2900 pmol/l and 3100 pmol/l; and/or in that said steps (a′), (b) and (c) are carried out by a computer.

36. The method according to claim 34, characterized in that said subject is in homeostatic imbalance.

37. The method according to claim 34, characterized in that said parameter V^{HT} in relation with the arterial hypertension status of said subject is function of the age of said subject, and is comprised between 0 and 11.

38. The method according to claim 34, wherein said parameter V^{HT} in relation with the arterial hypertension status of said subject is determined according to the following mathematical equation
V^{HT}=a(age)+b
wherein a is a variable in function of the age of said subject, with preference a is a value comprised between 0 and 10.0, and
wherein b is a variable in function of the seriousness of the arterial hypertension status, with preference b is a value comprised between 0 and 1.0.

39. The method according to claim 34, wherein the reference values used in step (b) in relation with AD1 are as followed:
with respect to the age of said subject, the minimum reference value is at least 50 years and/or the maximum reference value is at most 90 years,
with respect to the score of said subject to a questionnaire adapted for screening cognitive function, the minimum reference value is at least 14 and/or the maximum reference value is at most 29;
with respect to the dopamine concentration [D], the minimum reference value is at least 90 pmol/l, and/or the maximum reference value is at most 1000 pmol/l;
with respect to the adrenaline concentration [A] the minimum reference value is at least 40 pmol/l, and/or the maximum reference value is at most 2000 pmol/l,
with respect to the noradrenaline concentration [NA] the minimum reference value is at least 2900 pmol/l, and/or the maximum reference value is at most 9000 pmol/l.

40. The method according to claim 34, wherein the reference values used in step (b) in relation with AD2 are as followed:
with respect to the age of said subject, the minimum reference value is at least 45 years and/or the maximum reference value is at most 95 years,
with respect to the score of said subject to a questionnaire adapted for screening cognitive function, the minimum reference value is at least 3 and/or the maximum reference value is at most 30;
with respect to the dopamine concentration [D], the minimum reference value is at least 140 pmol/l, and/or the maximum reference value is at most 1000 pmol/l;
with respect to the adrenaline concentration [A] the minimum reference value is at least 80 pmol/l, and/or the maximum reference value is at most 1500 pmol/l,
with respect to the noradrenaline concentration [NA] the minimum reference value is at least 750 pmol/l, and/or the maximum reference value is at most 3000 pmol/l.

41. The method according to claim 34, wherein the reference values used in step (b) in relation with PSY are as followed:
with respect to the age of said subject, the minimum reference value is at least 35 years and/or the maximum reference value is at most 80 years,
with respect to the score of said subject to a questionnaire adapted for screening cognitive function, the minimum reference value is at least 21 and/or the maximum reference value is at most 30;
with respect to the dopamine concentration [D], the minimum reference value is at least 250 pmol/l, and/or the maximum reference value is at most 750 pmol/l;
with respect to the adrenaline concentration [A] the minimum reference value is at least 180 pmol/l, and/or the maximum reference value is at most 450 pmol/l,
with respect to the noradrenaline concentration [NA] the minimum reference value is at least 1000 pmol/l, and/or the maximum reference value is at most 4000 pmol/l.

42. The method according to claim 34, wherein the reference values used in step (b) in relation with DLB are as followed:
with respect to the age of said subject, the minimum reference value is at least 55 years and/or the maximum reference value is at most 85 years,
with respect to the score of said subject to a questionnaire adapted for screening cognitive function, the minimum reference value is at least 7 and/or the maximum reference value is at most 25;
with respect to the dopamine concentration [D], the minimum reference value is at least 150 pmol/l, and/or the maximum reference value is at most 400 pmol/l;
with respect to the adrenaline concentration [A] the minimum reference value is at least 80 pmol/l, and/or the maximum reference value is at most 700 pmol/l;
with respect to the noradrenaline concentration [NA] the minimum reference value is at least 900 pmol/l, and/or the maximum reference value is at most 8000 pmol/l.
